(19) United States
(12) Patent Application Publication
(10) Pub. No.: US 2004/0096930 A1
(43) Pub. Date: May 20, 2004

(54) Method for monitoring biotechnological processes

(76) Inventors: Bernhard Lendl, Wien (AT); Kurt Christian Schuster, Wien (AT)

Correspondence Address: RANKIN, HILL, PORTER & CLARK, LLP, 925 EUCLID AVENUE, SUITE 700, CLEVELAND, OH, 44115-1405, US

(21) Appl. No.: 10/473,929

(22) PCT Filed: Apr. 5, 2002
(86) PCT No.: PCT/AT02/00105

(30) Foreign Application Priority Data
Apr. 5, 2001 (AT) A 554/2001

(51) Int. Cl. C12M 1/34

(52) U.S. Cl. 435/29; 435/288.7

(57) ABSTRACT

The present invention relates to a method for monitoring biotechnological processes involving microorganisms, said method being characterized in that both the process medium and the microorganisms are directly spectroscoped in said medium by means of attenuated total reflection (ATR) at an ATR crystal whereby microorganisms come at least to a distance from the ATR crystal which corresponds to the evanescent field's depth of penetration, IR spectroscopy being carried out during this time interval, thereupon the microorganisms-containing medium being removed from the ATR crystal which then is washed and its contact is optionally renewed with the microorganisms-containing process medium, and moreover the invention relates to apparatus with which to carry out said method.

The invention relates to a method for monitoring bio-technological processes using microorganisms.

Monitoring bio-technological (fermentation) processes is exceedingly complex. Besides the substances produced and their content in the fermentation medium, the microorganisms and their physiological state also must be monitored. Data regarding the fermentation process, for instance yields, viability and metabolic state of the microorganisms should be sensed at least approximately in real time during such process monitoring in order to immediately react as needed to changes in the composition of the fermentation medium or in the microorganisms' state. Illustratively process control of the soluble components in reaction processes, particularly in fermentation processes using infrared (IR) spectroscopy, already has been described to measure substances such as glucose, acetic acid, lactic acid, lactose and galactose, also ethanol. This methodology also allows detecting several substances simultaneously.

Even though in principle it is feasible to carry out IR spectroscopy of biologically whole cells (see for instance Hopkinson et al, Analyst 112 (1987), pp 501-5; Fayolle et al, Vibr. Spectroscopy 14 (1997), pp 247-52), such a procedure has been considered to-date only in the dried state for instance in deuterium oxide for accurate cell analysis (McGovern et al, J. Biotechnol. 72 (1999), pp 157-67; Schuster et al, Vibr. Spectroscopy 19 (1999), pp 467-77 and Majara et al, J. Inst. Brew. 104 (1998), pp 143-6). As a result permanently monitoring cells in biological processes using whole-cell IR spectroscopy has not been considered practical both for equipment and procedural reasons. Again IR spectroscopy of whole cells has not been considered practical because of bio-fouling effects: when testing still non-desiccated solutions of fermentation containing cells, deposits will form on the IR test crystals (ATR crystals) that must be removed manually (Hopkinson et al loc. cit who also emphasized the problems of scratching the crystals and the ensuing contamination and generation of artifacts in the subsequent spectra). Such manual purification therefore is considered as prohibitively costly and inappropriate for routine analysis during process monitoring. On the other hand IR spectroscopy of dried cells is enormously time-consuming and entails undue preparation to turn the cells into an appropriate form for IR testing. It may be generally assumed that already specimen preparation shall require at least 25 minutes (Majara et al, loc. cit). Moreover appropriate on-line equipment with which to carry out the monitoring of biotechnological processes using microorganisms—where furthermore the composition and state of the microorganisms may be monitored (Majara et al, loc. cit), is unavailable.

The spectra taken by Fayolle et al were measured in an optical cell with a layer thickness of 38μ. Such a configuration is unsuitable for process control because susceptible to clogging as the measurement is repeated. Most significantly, the system described by Fayolle et al does not allow measuring the amide IR bands.

Another substantial drawback of the IR technology is the contamination of the test cells with polymeric or organic materials (including bio-fouling), in particular when the optical crystal of the in-line cells are coated irreversibly by the contaminants and can be rid of such coatings only by laborious physical cleaning (U.S. Pat. No. 5,604,132).

Accordingly the objective of the present invention is a method for monitoring biotechnological processes while using microorganisms, wherein both the soluble substances in the processing medium and the microorganisms may be monitored and tested rapidly, reliably, automatically, in well reproducible manner and with a high rate of information content.

Therefore the present invention offers a method of the above cited kind which is characterized in that both the processing medium and the micro-organisms in the processing medium are directly spectroscoped in the IR range by attenuated total reflections (ATR) at an ATR crystal, the processing medium containing the microorganisms making contact with the ATR crystal whereby the microorganisms reach at least a distance from the ATR crystal corresponding to the depth of penetration of the evanescent field, and spectroscopy in the IR range is carried out during this contacting the processing medium containing the microorganisms then being removed from the ATR crystal, said ATR crystal being washed and as called for a new contact shall be established with the micro-organisms containing processing material.

It was discovered in surprising manner that the method of the present invention allows fully and comprehensively monitoring biotechnological processes using IR spectroscopy, not only a plurality of soluble compounds being tested and monitored, but also the on-line state of the microorganisms and their composition being determined nearly in real time. This procedure of the invention may be carried out without drying the microorganisms and consequently the entire process may be carried out so quickly that it shall be suitable for on-line process control. The soluble components of the culture medium may be spectroscoped in conventional manner in the IR range for instance in an in-line cell. The microorganisms contained in the process medium are moved in a special contacting procedure at least to a distance from the ATR crystal which corresponds to the depth of penetration of the evanescent field and are accurately spectroscoped in the IR range during this contact procedure.

The penetration depth of the evanescent field is a special value depending on material, wavelength, angle of incidence and media that was defined for instance in Harrick (J. Opt. Soc. Am. 55 (7), 1965, pp 851-7). The depth of penetration=distance from the surface of the ATR crystal at which the evanescent wavelength amplitudes drops to one half the value said wave exhibits at the surface. As regards diamonds, aqueous solutions a wave number of 1,000, said depth is about 2μ.

Thereupon the microorganisms-containing process medium is removed from the ATR crystal which next is washed with aqueous solutions and optionally new contacts by means of the microorganisms-containing process medium may be carried out.

Preferably the IR spectroscopy shall be Fourier-Transform (FTIR spectroscopy) which, as already mentioned, was used in the past to monitor biological processes, however solely with dissolved substances. Such determinations using IR lasers (for instance quantum cascade lasers as the IR source also are advantageous.

A diamond is preferably used as the ATR crystal within the scope of the method of this invention. Foremost as regards washing, said diamond is substantially more stable than are conventional ATR crystals and it is particularly advantageous in ATR measurements. Preferably the ATR measurement is carried out using a planar waveguide as the ATR crystal, in particular using the methodology described by Braiman et al, Appl. Spectrosc. 51 (4), 1977, pp 592-7 (also see WO 00-36442 A, U.S. Pat. No. 5,980,831 A).

The process medium shall preferably be moved through an automated flow system from the bio-reactor wherein the biological process is being carried out to the ATR crystal, and during the time during which the bio-suspension flows over the ATR crystal (“flow on”), the “large” microorganism cells will not come near the ATR surface and therefore are not sensed, or only insignificantly, by the IR light. Therefore those cells do not significantly contribute to the measured spectra and only the dissolved components such as sugars, alcohols, amino acids etc. are. determined quantitatively during the flow-on phase, in the manner described in Kansiz et al, Analytical Chimica Acta 21149 (2001), pp 1-12. Illustratively in that instance, acetone, acetate, n-butanol, butyrate and glucose were measured at middle IRs when monitoring an acetone-butanol fermentation.

If for instance the flow is stopped, the microorganisms shall deposit on the ATR crystal, and said organisms then may be measured. In some cases mechanical pressures also may be applied to render more efficient the approach of the microorganisms to the ATR crystal.

However the approach of the microorganisms to the ATR crystal (and hence the temporary measurement of said microorganisms) may be implemented in a different way, for instance by temporarily retaining the micro-organisms in the process solution using appropriate filters and optionally by said organisms being moved nearer by means of centrifugal force, by biolectrical/magnetic apposition to the ATR crystal etc.

The measurement of the cells also may involve varying the angle of incidence at the interface of ATR crystal and medium in the manner described by Shick et al (Appl. Spectrosc. 47 (8) 1993, pp 1237-44). However the measurement also may be carried out using two or more ATR crystals, optionally at different angles of incidence.

In the present invention, washing preferably is carried out by treating the ATR crystal with a base, for instance using 1M NaOH, and then rinsing it with distilled water. The addition of bases allows completely deprotonating the analyzed acids and stopping metabolic activities. Alternatively to or in combination with the above steps, alternative wash solutions may also be used, for instance Na₂ CO₃ (for instance 5%), again preferably followed by washing phases with distilled water.

It was discovered in the present invention that the duration of a measurement cycle of whole cells using IR may be lowered from at least nearly less than 30 minutes in conventional operation (Majara et al, loc. cit) preferably to less than 20 minutes and in especially preferred manner to less than 10, in particular less than 5 minutes, making possible monitoring times of the biological process at the stated time intervals and thereby allowing to monitor the process in adequately accurate manner.

The method of the present invention is especially well suited to monitor biological processes using unicellular eukaryotes, in particular yeasts or bacteria in particular E. coli, the method however being applicable in principle to any suspension-(submersion) cultivation, i.e., for which microorganisms or their aggregates are in suspended form.

Accordingly especially preferred microorganisms are recombinant ones and or those producing antibiotics that on account of their sensitivity and their complex metabolic processes require an exceedingly complex process analysis. The method of the present invention also may be used in the preparation of unicellular protein or in the preparation of foodstuffs or nutrients based on microorganisms, further to measure microbial carbohydrates.

Accurate process control also is required in those biotechnological processes wherein inclusions will form in the microorganisms. Again these microorganisms or inclusions may be well monitored using IR spectroscopy and also may be controlled on an industrial scale using the method of the present invention. It is known that the secondary protein structure can be analyzed using IR spectroscopy (Millot et al, Anal. Chim. Acta 295 (1994) pp 233-41; Naumann et al, “Infrared and Raman Spectroscopy of Biological Materials” (2000), pp 323-378; Eds. Gremlich et al, Marcel Decker, New York). Straight inclusions exhibit a characteristic secondary structure particular rich in β-sheet.

The method of the present invention is applicable over the full IR range, but preferably the spectroscopy shall take place in the middle IR range at a wave number between 4,000 and 400/cm, in particular between 1,800 and 900/cm, in the so-called fingerprint range. However measurement may also and obviously be carried out in the near IR range, that is at wavelength between 2.5 and 750μ.

The spectroscopy of soluble ingredients shall preferably be carried out in the process medium before the spectroscopy of the microorganisms is. Microorganism spectroscopy shall preferably be followed by the washing stage.

As already mentioned above, the method of the present invention preferably is carried out in such a way that following the contact between the ATR crystal and the process medium, the motion of said medium shall be stopped and as a result the microorganisms shall be deposited on said ATR crystal (whereby they also may come appropriately near the ATR crystal), whereafter microorganisms spectroscopy shall be carried out.

The method of the present invention allows monitoring the secondary structure of proteins in the process medium, in particular also as regards the microorganisms. The lipid content in the process medium and/or for the microorganisms also may be monitored.

In another respect the present invention also relates to apparatus with which to carry out its method and comprising an ATR element fitted with an ATR crystal and connected to an automated flow system, further an IR spectroscopic device connected to the ATR crystal and a washing system connected through an automated flow system to the said ATR element.

Because of the automated flow system, said apparatus is especially well suited for IR control and the monitoring of biotechnological processes. In general an automated flow system moves and handles the illustratively liquid or suspended specimens by means of pumps, hoses and valves. Preferably the IR spectroscopic device is connected by an electronic data processing system (IR EDP) analyzing the recorded spectra and by means of which preferably process components (adjusting elements) such as feed devices, temperature regulators, pH regulator etc. for the bioreactor may be controlled by the IR EDP when connected to it, allowing controlling a change in process parameters In this manner automated process control may be carried out for instance to efficiently and automatically monitor, control and, as called for, correct predetermined reference values by the controlled feeding of foodstuffs, by means of process control elements, into the bioreactor. Illustratively, based on the spectroscopic IR data, one may add foodstuffs and also for instance expression repressors, attenuators or inductors (for instance in recombinant cultures).

Preferably the apparatus of the invention also comprises a bioreactor which may be filled as called for with process medium and microorganisms.

Also conditioning of the process medium may be carried out by means of the automated flow system, or one might proceed to measure, adjust the pH values, temporarily filter or separate in stages, to separate substances that might degrade the measurement, etc.

Preferably the flow system of the present invention comprises hoses having an inside diameter larger than 0.7 mm, that is larger than the standardized diameter (0.3, 0.5 and 0.7 mm) because hoses of standard diameters may easily be clogged on account of the microorganisms. Preferably the hoses of the invention exhibit inside diameters of 1 mm and larger, in particular 1.2 mm and (much) beyond.

Even though in normal IR spectroscopic operation using ATR crystals a small volume of detection (=dead volume of the ATR element; measurement volume on the ATR crystal) is desired (lesser quantity of specimen, more accurate measurement, reduced dilutions etc.), it was found advantageous as regards the apparatus of the invention to keep the dead volume of the ATR element larger than the conventional magnitude of 5 μltr. Therefore the dead volume in the apparatus of the present invention is preferably larger than 5 μltr, and in especially preferred manner it is 10 μltr and in particular 20 μltr or more.

Preferably the IR spectroscopic device is a FTIR spectrometer which is especially well suited to secure optimal IR spectra using ATR crystals. Again IR measurements using lasers (for instance quantum cascade lasers) will be advantageous.

Preferably the ATR crystal shall be configured horizontally opposite the process medium which shall be measured and which flows past it, whereby the process medium can be guided over the ATR crystal. Once the flow is stopped, the microorganisms by gravity alone may settle on the ATR crystal and be measured there. Illustratively however the ATR crystal also may be fitted with a microorganisms collector such as folding filter which, when said microorganisms should be measured, will unfold upward and thus shall position the “trapped” microorganisms on the ATR crystal.

Moreover the microorganisms may be moved toward the ATR crystal by using appropriate electrical charge induction on said organisms. Illustratively this phenomenon may be implemented by an external electric field in the manner of “free-flow electrophoresis” (Raymond et al, Anal. Chem. 68 (15), 1996, pp 2515-22) or also by depositing a charged surface, for instance a layer of poly-L-lysine, on the ATR crystal.

Preferably furthermore substances may be fitted on the ATR crystal to favorably affect the washing procedure or the adhesion of microorganisms, and in particular such substances shall assume the configuration of monolayers.

Preferably the ATR element is designed as an in-line cell, as a result of which the measurement of soluble components/cells may be implemented by a combination of “flow on” and “stop” stages.

As already mentioned above, said ATR crystal preferably shall be a diamond which on account of its hardness and the associated low maintenance is especially well suited to said method of the present invention. Germanium or silicon crystals, or crystals of comparable hardness, also are appropriate as ATR crystals.

Preferably the apparatus of the present invention shall be fitted with a retaining device to stop the process medium's flow, where said device nevertheless allows IR measurements.

The present invention is elucidated below by means of the illustrative embodiments described herein and in relation to the appended drawings, but is not restricted to them.

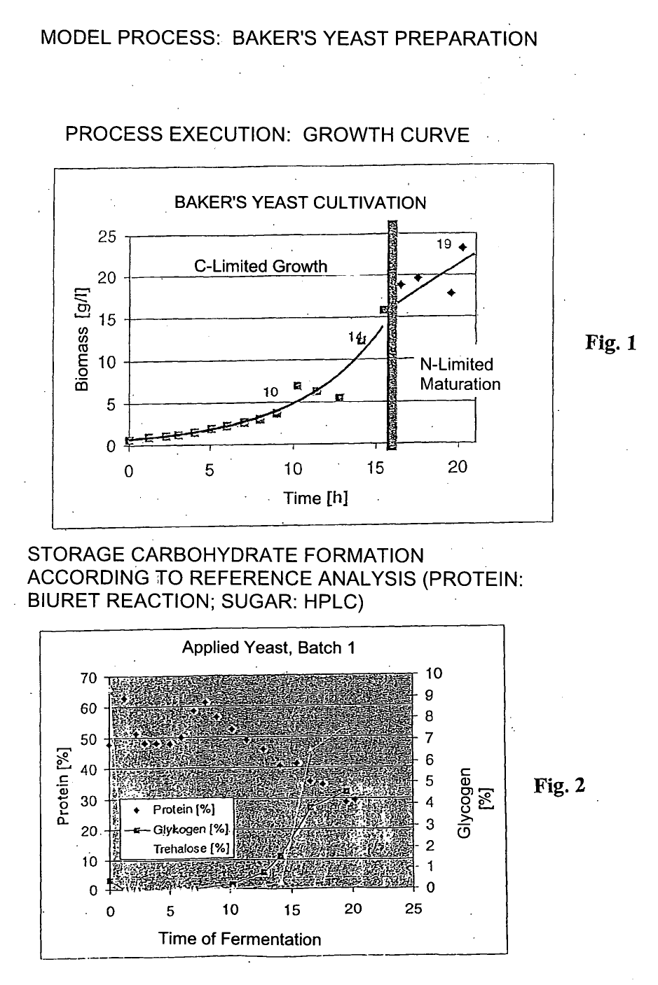

FIG. 1 shows process implementation and growth curve of baking yeast production,

FIG. 2 shows the formation of storage material in this fermentation,

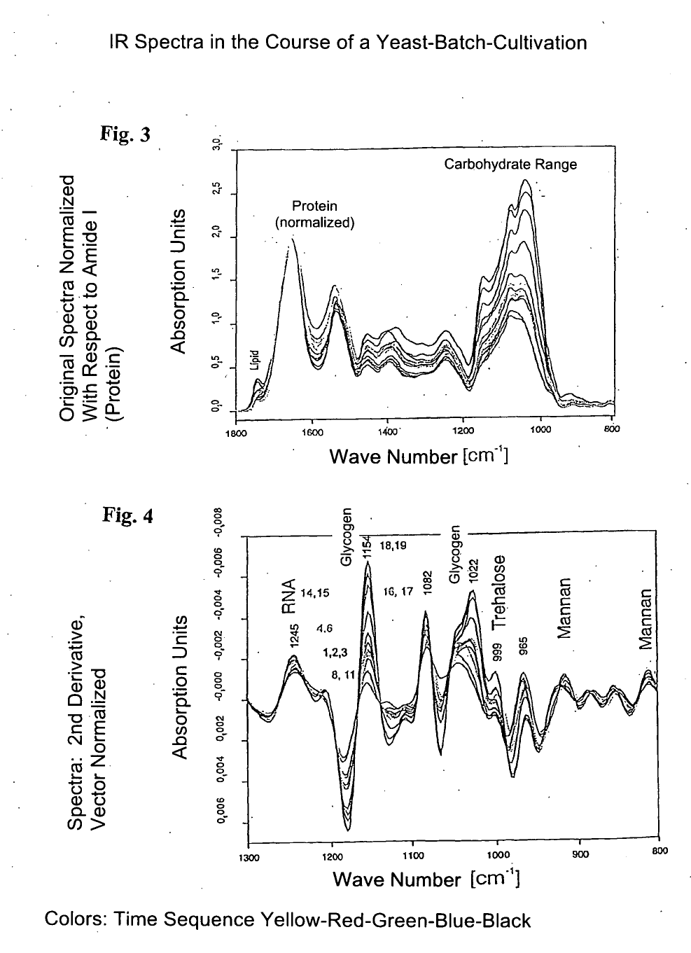

FIG. 3 shows an IR spectrum of dried baking yeast normalized with respect to amide (protein),

FIG. 4 shows the second derivative of FIG. 3 in vector-normalized form,

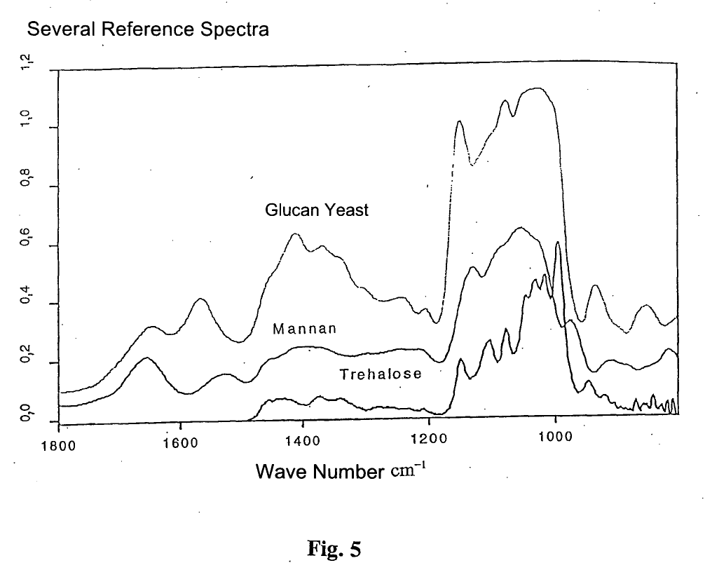

FIG. 5 shows reference spectra of pure cell components,

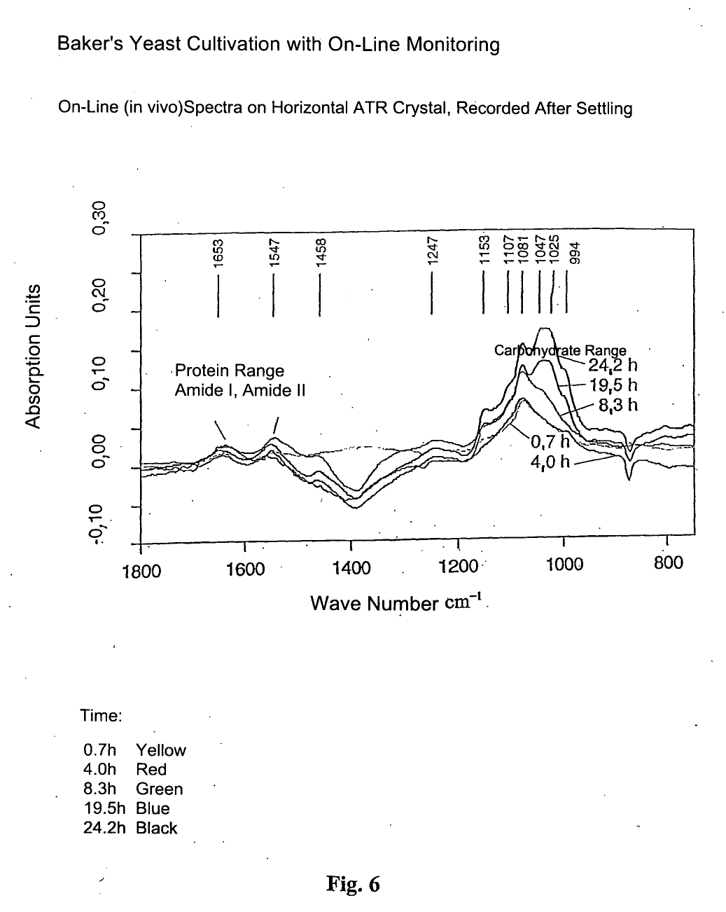

FIG. 6 shows in vivo on-line spectra, baking yeast fermentation recorded using the ATR technique,

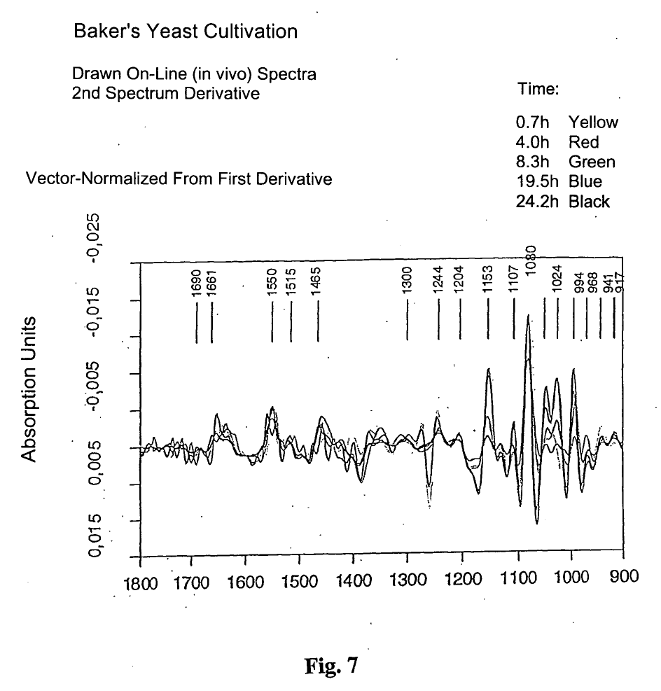

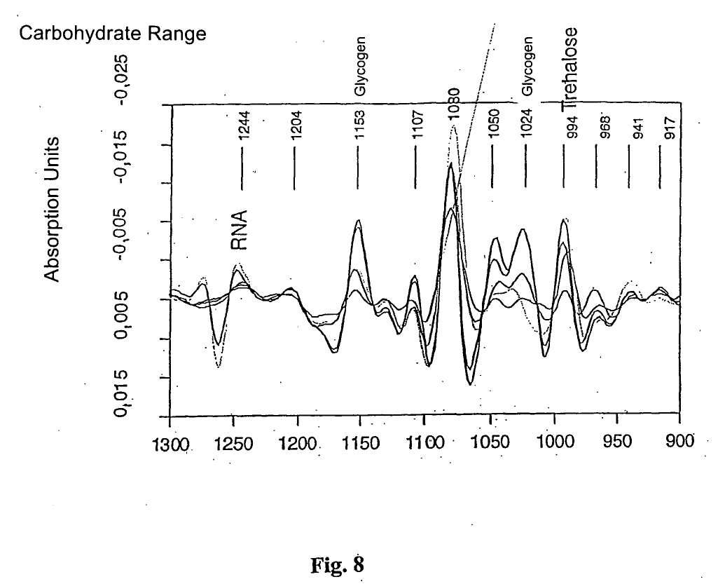

FIGS. 7, 8 are in vivo, on-line microorganisms spectra of the invention, and

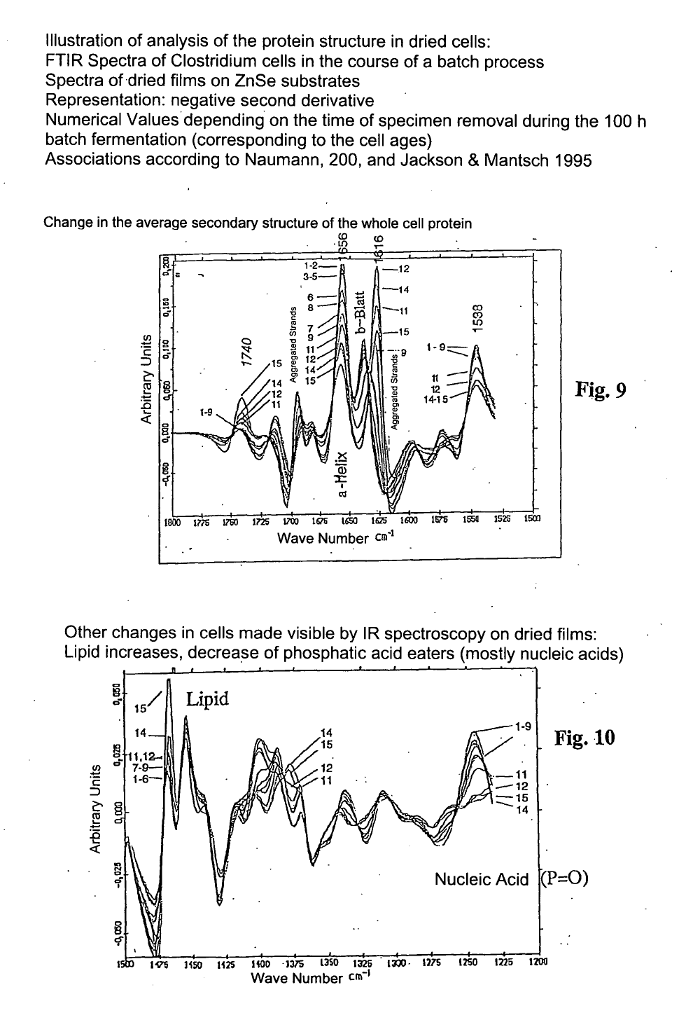

FIGS. 9, 10 show FTIR spectra of cells (chlostridium) in the dried state; FIG. 9 shows the analysis of different secondary structures in the proteins of dried cells.

### EXAMPLES

The efficiency of the method of the invention was displayed in relation to yeast fermentation:

First a yeast culture was kept for about 15 h at C-limited growth and then in an N-limited maturation phase. No storage carbohydrates are included into the yeast cells during the C-limited growth phase. Inclusion only occurs after switching to the N-limited growth phase. FIG. 2 shows the results of reference analyses (HPLC, wet-chemical).

This biotechnological process was run twice: as regards the first biotechnological process (of the state of the art), specimens were removed manually, dried and spectroscoped. The results are shown in FIGS. 3 and 4. They clearly show that carbohydrates are only absorbed in the N-limited phase. The information contained in the middle IR also allows distinguishing between different sugars. The sequence of storage material formation of glycogen first, then trehalose, could be corroborated by external reference analysis (FIG. 2) and reference spectra of pure cell components (FIG. 5).

The method of the present invention was applied to the second biotechnological process. The microorganism spectra measured during the “stopped flow” time interval are very similar (FIGS. 6, 7 and 8) to those recorded in the dry state. Comparison of the second derivatives clearly shows that the on-line recorded spectra practically contain the same information as those of the dried microorganisms (FIGS. 4 and 8).

These experiment therefore are evidence of the effectiveness of the method of the present invention. As shown by FIGS. 9 and 10, information may be derived from the spectra also regarding the secondary structure of proteins as well as the increase/decrease of lipids, RNA and the like. The method and the apparatus of the present invention may be used for process control/monitoring regarding any cultivation process of suspension cultures. The invention also allows detecting on-line the degeneration of microorganisms. Especially preferred industrial fields of application for the method of the invention are the preparation of recombinant proteins, in particular of proteins formed intracellularly, especially when forming inclusion bodies, the preparation of antibodies, the preparation of polyhydroxyalkanoates, moreover in the field of basic bioprocess research.

1. A method for monitoring biotechnological processes using microorganisms, characterized in that
both the process medium and the microorganisms in this medium are directly spectroscoped by means of attenuated total reflection (ATR) at an ATR crystal in the infrared (IR) range, the microorganisms-containing process medium being made to contact the ATR crystal in a manner that microorganisms shall arrive at least within such a distance from the ATR crystal which corresponds to the depth of penetration of the evanescent field and that spectroscopy is carried out in the IR range during this contact time interval, whereupon the microorganisms-containing process medium shall be removed from the ATR crystal and optionally new contact with the microorganisms-containing process medium is carried out.

2. Method as claimed in claim 1, characterized in that the IR spectroscopy is carried out using Fourier Transform IR spectroscopy (FTIR spectroscopy).

3. Method as claimed in either of claims 1 and 2, characterized by using a diamond ATR crystal.

4. Method as claimed in one of claims 1 through 3, characterized in that the process medium is moved by an automated flow system out of the biotechnological-process bioreactor to the ATR crystal.

5. Method as claimed in one of claims 1 through 4, characterized in that the washing procedure includes treating the ATR crystal with a base.

6. Method as claimed in one of claims 1 through 5, characterized in that the spectroscopy is carried out at intervals of at least 20 minutes, preferably at least 10 minutes, in particular at least 5 minutes.

7. Method as claimed in one of claims 1 through 6, characterized in that unicellular eucaryotes, in particular yeasts, or bacteria, in particular E. coli, are used as microorganisms.

8. Method as claimed in one of claims 1 through 7, characterized in that the spectroscopy is carried out at a wave number of 400 to 4,000.

9. Method as claimed in one of claims 1 through 8, characterized in that the spectroscopy of soluble components in the process medium is carried out before the spectroscopy of microorganisms.

10. Method as claimed in one of claims 1 through 9, characterized in that after the ATR crystal has been contacted by the process medium, said medium's motion is stopped, whereby the microorganisms are deposited on the ATR crystals and then the microorganisms' spectroscopy is carried out.

11. Method as claimed in one of claims 1 through 10, characterized in that the microorganisms used are recombinant microorganisms and/or antibiotics-producing microorganisms.

12. Method as claimed in one of claims 1 through 11, characterized in that the biotechnological process is carried out while forming inclusion bodies in the microorganisms.

13. Method as claimed in one of claims 1 through 12, characterized in that the secondary structure of proteins is monitored in the process medium.

14. Method as claimed in one of claims 1 through 13, characterized in that the lipids content in the process medium is being monitored.

15. Apparatus to carry out a method as claimed in one of claims 1 through 14, comprising an ATR element fitted with an ATR crystal and connected to an automated flow system, further an IR spectroscopic device connected to the ATR crystal and a washing system connected by an automated flow system to the ATR element.

16. Apparatus as claimed in claim 15, characterized in that the IR spectroscopic device is connected to an electronic data processing system (IR EDP) to analyze the recorded spectra.

17. Apparatus as claimed in claim 15, characterized in that the apparatus further comprises a bioreactor optionally filled with a process medium containing microorganisms.

18. Apparatus as claimed in claim 17, characterized in that the IR EDP is connected by process control devices to the bioreactor, said devices being controlled by the IR EDP.

19. Apparatus as claimed in one of claims 15 through 18, characterized in that the IR spectroscopic device is an FTIR spectrometer.

20. Apparatus as claimed in one of claims 15 through 19, characterized in that the ATR crystal is configured horizontally opposite the process medium so that said medium can be made to pass over the ATR crystal.

21. Apparatus as claimed in one of claims 15 through 20, characterized in that the ATR element is an in-line cell.

22. Apparatus as claimed in one of claims 15 through 21, characterized in that the ATR crystal is a diamond.

23. Apparatus as claimed in one of claims 15 through 22, characterized in that the automated flow system comprised hoses having an inside diameter exceeding 0.7 mm, preferably 1.0 mm or more and in particular 1.2 mm or more.

24. Apparatus as claimed in one of claims 15 through 22, characterized in that the dead volume of the ATR element is larger than 5 μltr, preferably larger than 10 μltr or more and in particular 20 μltr or more.

25. Apparatus as claimed in one of claims 15 through 24, characterized in that the IR spectroscopic device comprises a laser source as the IR source.
